(19) United States
(12) Patent Application Publication
(10) Pub. No.: US 2026/0345249 A1
(43) Pub. Date: Nov. 5, 2026

(54) FINGERTIP-MOUNTED ULTRASOUND IMAGING DEVICE WITH ADJUSTABLE TRANSDUCER ORIENTATION FOR DYNAMIC SCANNING PLANE CONTROL

(71) Applicant: Charles Y. Y. Cheng, Vancouver (CA)

(72) Inventor: Charles Y. Y. Cheng, Vancouver (CA)

(21) Appl. No.: 19/426,107

(22) Filed: Dec. 19, 2025

Related U.S. Application Data
(60) Provisional application No. 63/799,693, filed on May 4, 2025.

(51) Int. Cl. A61B 34/20; A61B 8/00; A61B 8/08

(52) U.S. Cl. CPC A61B 34/20; A61B 8/0841; A61B 8/4227; A61B 8/4427; A61B 8/461; A61B 8/56; A61B 2034/2063

(57) ABSTRACT

A fingertip-mounted variable axis ultrasound imaging device and system are disclosed for performing real-time ultrasound-guided medical procedures. The device includes a compact housing worn on a ventral surface of a user's finger, an adjustable mounting joint coupled to the housing, and an ultrasound transducer operatively coupled to the adjustable mounting joint. The adjustable mounting joint permits pivotable, tiltable, or rotatable movement of the transducer in spatial axes, allowing the scanning plane to be dynamically altered. The ultrasound transducer emits ultrasound waves into tissue and receives echo signals that are processed to generate real-time images on a display device. During operation, the user holds a medical device, such as a syringe, cannula, catheter, or biopsy needle, in a second hand while the first hand supports and positions the fingertip-mounted ultrasound device over a patient's target site.

### CROSS-REFERENCE TO RELATED APPLICATION

This application claims the benefit of U.S. Provisional Patent Application Ser. No. 63/799,693 filed on May 4, 2025, which is incorporated herein by reference in its entirety.

### FIELD OF THE INVENTION

The present disclosure generally relates to the field of medical imaging devices, and more particularly to miniaturized ultrasound transducers configured for use in ultrasound-guided medical procedures. Specifically, the disclosure pertains to a fingertip-mounted ultrasound imaging apparatus with an adjustable orientation mechanism for dynamic scanning plane control and improved procedural ergonomics.

### BACKGROUND

Ultrasound imaging is widely used in medical diagnostics and interventional procedures to visualize, in vivo and in real-time, soft tissue, including dermis and subcutaneous anatomical structures such as vascular, neurological, muscular, connective, and osseous structures. In many clinical scenarios, such as filler implants, neuromodulator injections, biostimulator placements, vascular access, regional anaesthesia or pain management, joint and tendon interventions, fluid drainage, and tissue biopsies, ultrasound guidance is essential for accurate needle, catheter, or cannula placement.

Conventional ultrasound systems typically employ handheld probes that are coupled to a console or portable device via a cable or wireless. While these handheld probes provide adequate image quality, they are relatively bulky and require the operator to use one hand to hold the probe and the other to manipulate the medical device (e.g., a syringe or catheter). This configuration can be ergonomically challenging, particularly in confined or awkward anatomical regions such as the face, neck, wrist, or ankle. Performing a procedure under ultrasound guidance requires exact and stable probe placement. Maintaining stable and appropriate probe contact with the skin while advancing a needle or cannula often requires either an assistant or the use of improvised positioning techniques, which can compromise ergonomical position, procedural precision, and sterility.

Various compact and “wearable” ultrasound probes have been proposed in recent years to improve workflow efficiency. For example, finger-mounted or ring-type ultrasound transducers have been developed to enable single-operator control. However, most of these devices feature fixed-axis transducer orientations, typically aligned parallel to the operator's finger. Such designs restrict the scanning plane to a single direction relative to the finger, making it difficult to adjust the imaging angle and plane to optimally meet the needs of the assessment or procedure. Prior devices with fixed transducer orientations often force the operator to perform awkward wrist repositioning and prevent maintaining an ergonomically optimal hand posture during imaging or instrumentation.

As a result, clinicians often must reposition their wrist, hand, or the entire arm to obtain a suitable imaging plane. In ultrasound-guided procedures, when the scanning and procedure hands are combined, the positions of the hands and wrists are often ergonomically suboptimal, increasing operator difficulty and decreasing procedural accuracy.

Furthermore, fixed-orientation fingertip probes do not accommodate the wide variation in medical devices used, patient positioning, patient target tissue orientation, operator posture, and approach angles that occur in different interventional procedures. The inability to dynamically alter the probe orientation limits the device's adaptability across these contexts.

Existing ultrasound systems also face challenges in ergonomics and cable management. Rigid or heavy cable connections can interfere with fine hand movements and cause drag on the probe during needle advancement. Moreover, the lack of an integrated support mechanism for maintaining the transducer's position relative to the operator's finger leads to unstable imaging contact, particularly when the clinician's hand is partially occupied with other instruments.

In a fixed transducer to finger scenario, the size and shape of the transducer is often limited to the tip of the finger. In a curved transducer design, the image can potentially be distorted and in a liner design, the field of view is often narrow. With our variable angle and plane design, the size of the transducer can be increased.

Accordingly, there remains a need for an improved fingertip-mounted ultrasound imaging device that provides dynamic control of transducer orientation, maintains stable skin contact, and supports ergonomic, single-operator use during ultrasound-guided procedures. Such a device should enable clinicians to dynamically adjust the scanning plane based on the patient's position, the operator's hand orientation, and the type of medical device being used, without requiring substantial wrist or arm repositioning. There is also a need for a design that increases transducer stability and enhances procedural ease to reduce variability in imaging quality and operator fatigue.

### SUMMARY

The present disclosure provides a fingertip-mounted ultrasound imaging device, system, and method for performing real-time ultrasound-guided medical procedures. The invention is directed toward improving procedural ergonomics, imaging control, and single-operator efficiency in diagnostic and interventional ultrasound applications.

In one embodiment, the fingertip-mounted ultrasound imaging device includes a compact housing configured to be worn on a ventral surface of a user's finger, such as the index or middle finger. The housing is contoured for ergonomic comfort and secured to the finger using a fastening mechanism, such as a strap, loop, or band. The device further includes an adjustable mounting joint operatively coupled to the housing and an ultrasound transducer coupled to the adjustable mounting joint. The adjustable mounting joint is configured to permit pivotable, tiltable, and/or rotatable movement of the transducer relative to the finger, thereby enabling variable scanning plane orientation that can be dynamically altered as needed.

The ultrasound transducer is configured to emit acoustic waves into biological tissue and receive corresponding echo signals. These echo signals are processed by an associated computing and display system to generate real-time ultrasound images that visualize various tissue structures and procedural medical devices such as needles or cannulas. The adjustable mounting joint allows the transducer to maintain consistent skin contact and requires optimal imaging alignment across different anatomical surfaces and patient positions.

In certain embodiments, the ultrasound transducer may comprise a linear, convex, or curved probe array. The transducer supports advanced imaging modes, including but not restricted to Doppler imaging, beam steering, and compound imaging.

In another aspect, the invention provides an ultrasound imaging system comprising the fingertip-mounted ultrasound imaging device, a power and data module configured to supply electrical drive signals to the transducer and receive echo signals, and a computing device configured to process and render the ultrasound images on a display unit. The processing unit may include a smartphone, tablet, computer, or a standalone ultrasound console. The display unit may include a smartphone, tablet, computer monitor, or a standalone ultrasound console. In certain implementations, the display may be integrated into an augmented reality (AR) headset, allowing direct image projection into the clinician's field of view. This will allow the user to focus on the injection site while simultaneously viewing ultrasound guidance and the location of the medical device, such as a needle or cannula, below the skin surface. This will allow the user to easily view both the injection site and the ultrasound guidance image without looking away from the patient at a separate display unit.

In yet another embodiment, the invention provides an ultrasound-guided procedural system comprising the fingertip-mounted ultrasound imaging device and a medical device such as a syringe, cannula, catheter, or biopsy needle. During use, a clinician wears the fingertip-mounted ultrasound device on a finger of a first hand and holds the medical device in a second hand, while supporting and stabilizing or stretching the skin surface or injection entry point between the thumb and middle finger of the first hand. The index finger, fitted with the fingertip-mounted device, positions the ultrasound transducer over a target site on a patient. The system enables the clinician to observe real-time ultrasound images while simultaneously manipulating the needle or instrument, ensuring accurate placement at the correct tissue depth or target region beneath the skin. Optionally, the user may choose to mount the transducer on the middle finger of the first hand while using the thumb and index finger of the same hand for skin stabilization. The remaining fingers and the palm of the first hand can provide stable support for positioning the scanning finger and assisting the thumb and fingers.

A corresponding method of performing an ultrasound-guided medical procedure is also disclosed. The method includes wearing the fingertip-mounted ultrasound imaging device on an index finger of a first hand, holding a medical device in a second hand, supporting the medical device with a thumb and middle finger of the first hand, positioning the ultrasound transducer over a target site, operating the transducer to generate a real-time ultrasound image, and positioning the needle of the medical device at the target site based on the live image. The adjustable mounting joint of the fingertip-mounted device enables continuous optimization of the scanning plane during the procedure without disrupting hand stability or imaging contact.

The disclosed system and method provide numerous advantages over conventional ultrasound-guided setups. The fingertip-mounted configuration allows a single operator to perform both imaging and needle manipulation without assistance. The adjustable joint provides superior control of the imaging angle, enabling alignment with the needle trajectory or anatomical structures of interest. The compact, ergonomic design increases transducer stability, increases optimization of scanning angles and planes, reduces operator fatigue, improves ergonomics, improves coordination, and enhances safety during procedures that require high precision.

The fingertip-mounted ultrasound imaging device is adaptable for diverse medical applications, including vascular access, regional anaesthesia, musculoskeletal injections, abscess drainage, biopsies, and targeted drug or implant delivery. The device's modular construction accommodates interchangeable probe types, variable scanning depths, and optional wireless communication for real-time image transmission, making it suitable for point-of-care, bedside, and field use.

The fingertip-mounted ultrasound imaging device, associated system, and associated method of the present disclosure overcome one or more of the shortcomings of the prior art. Additional features and advantages may be realized through the techniques of the present disclosure. Other embodiments and aspects of the disclosure are described in detail herein and are considered a part of the claimed disclosure.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1A illustrates an example environment for performing ultrasound imaging, in accordance with an exemplary embodiment of the present disclosure;

FIG. 1B illustrates another example environment for performing ultrasound imaging, in accordance with an exemplary embodiment of the present disclosure;

FIGS. 2A-2C illustrate example configurations of a fingertip-mounted ultrasound imaging device worn on a finger in accordance with some embodiments of the present disclosure;

FIGS. 2D-2F illustrate additional embodiments and operational configurations of the fingertip-mounted ultrasound imaging device in accordance with some embodiments of the present disclosure;

FIG. 3 illustrates an example operational configuration demonstrating how a clinician employs the fingertip-mounted ultrasound imaging device in coordination with the medical device 104, in accordance with some embodiments of the present disclosure;

FIG. 4 illustrates another embodiment depicting the use of a fingertip-mounted ultrasound imaging device during an ultrasound-guided injection procedure in accordance with some embodiments of the present disclosure; and

FIG. 5 illustrates an example method for performing an ultrasound-guided medical procedure utilizing a fingertip-mounted ultrasound imaging device in accordance with some embodiment of the present disclosure.

Like reference numerals refer to like parts throughout the several views of the drawings.

### DETAILED DESCRIPTION

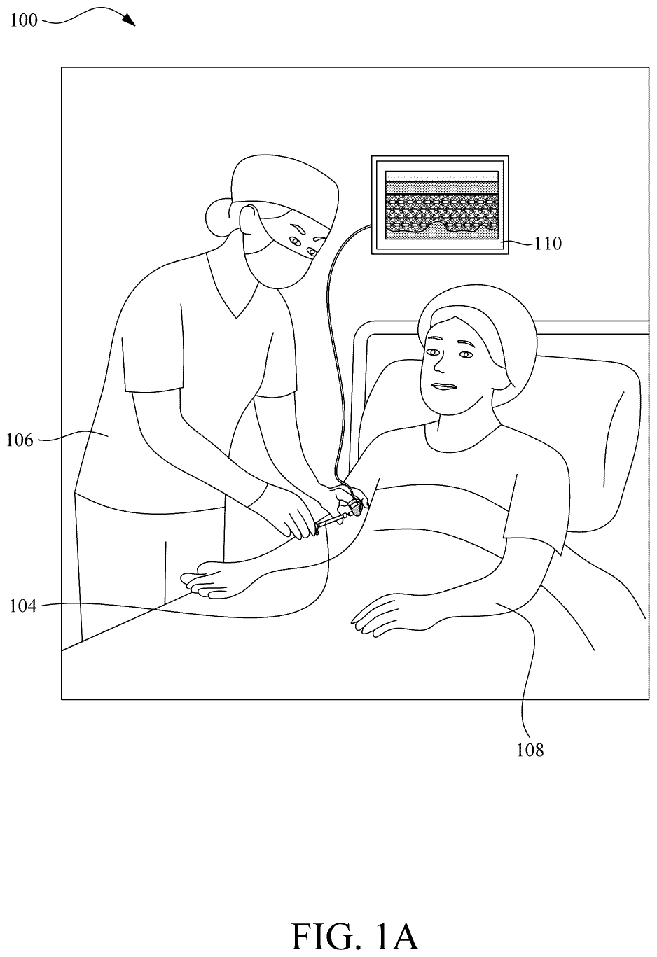

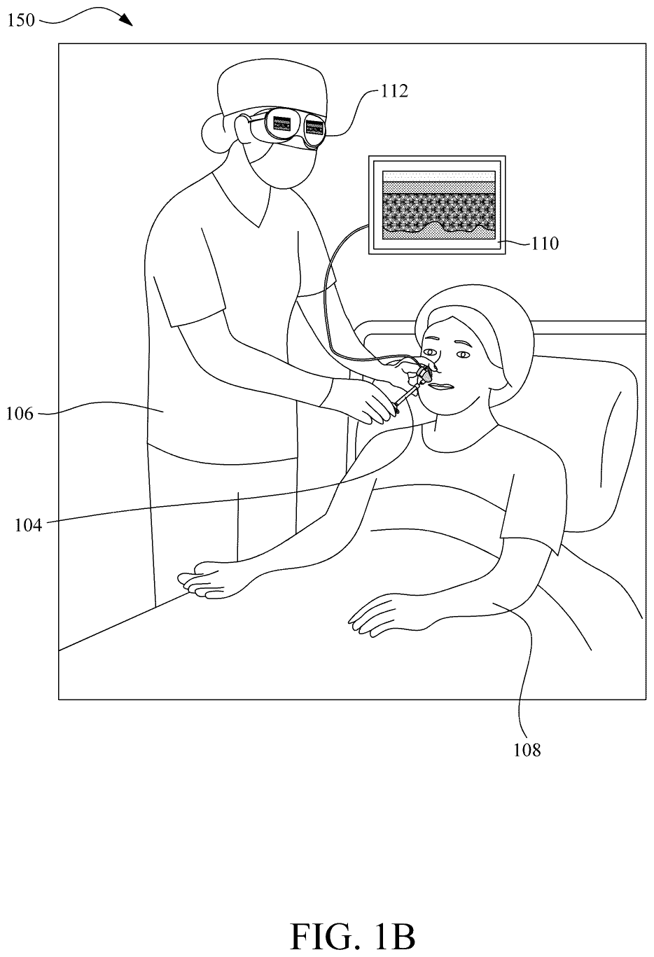

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Unless the context requires otherwise, throughout the specification and claims which follow, the word “comprise” and variations thereof, such as, “comprises” and “comprising” are to be construed in an open, inclusive sense, that is as “including, but not limited to.”

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

The headings and Abstract of the disclosure provided herein are for convenience only and do not interpret the scope or meaning of the implementations.

FIG. 1A illustrates an example environment 100 in which a fingertip-mounted ultrasound imaging device 102 is utilised by a health care professional 106 to perform an ultrasound-guided medical procedure on a patient 108. The example environment 100 further includes a medical device 104, such as a syringe, cannula, catheter, or biopsy needle, and a display 110 coupled to the fingertip-mounted ultrasound imaging device 102 for visualisation of real-time ultrasound images.

The fingertip-mounted ultrasound imaging device 102 may include a compact housing configured to be worn on one of the user's fingers, such as the index or middle finger, enabling ergonomic control and precision during medical interventions. The housing may include an adjustable mounting joint that supports a miniature ultrasound transducer. The adjustable mounting joint permits pivotable, tiltable, and/or rotatable movement of the transducer relative to the user's finger, thereby providing a variable scanning plane that can be dynamically altered without repositioning the user's hand or wrist.

In some embodiments, the ultrasound transducer of the fingertip-mounted ultrasound imaging device 102 may be a linear, convex, or curved array, capable of operating at frequencies ranging from about 7 MHz to about 50 MHz. The ultrasound transducer may be configured to emit ultrasound waves into the underlying tissue and to receive reflected echo signals, which are converted into real-time ultrasound images displayed on the display 110. The fingertip-mounted design allows the health care professional 106 to maintain tactile feedback and fine motor control during delicate procedures, such as injections, aspirations, or catheter insertions, while maintaining continuous visualization of the target tissue or structure.

The medical device 104, illustrated in FIG. 1 as a syringe, may include a syringe barrel, a plunger, and a needle or cannula configured for injection or aspiration. The syringe 104 may alternatively represent other interventional instruments, such as a catheter, a biopsy device, or a delivery system for therapeutic or diagnostic agents.

In use, the health care professional 106 may hold the syringe 104 in one hand (the “second hand”), while the fingertip-mounted ultrasound imaging device 102 is worn on a finger of the other hand (the “first hand”). The thumb and middle finger of the first hand may support or stabilize the transducer against the skin, while the index finger of the same hand positions the fingertip-mounted ultrasound imaging device 102 over the skin surface of the patient 108 near the target site. This configuration allows simultaneous imaging and manipulation of the syringe 104 for accurate needle placement.

The health care professional 106 may be any trained clinician, physician, nurse, or technician who performs ultrasound-guided diagnostic or therapeutic procedures. The fingertip-mounted ultrasound imaging device 102 enables the health care professional 106 to operate in a more natural, intuitive posture by combining tactile and visual feedback. The usage of the fingertip-mounted ultrasound imaging device 102 reduces the need for large handheld probes that occupy one entire hand and limit dexterity.

For example, during a vascular access procedure, the health care professional 106 may position the ultrasound transducer of device 102 over the patient's forearm, identify a vein using real-time ultrasound imaging, and simultaneously guide the needle of syringe 104 toward the identified vein while continuously monitoring needle trajectory on display 110. This integrated approach minimizes procedural time and improves accuracy compared to traditional ultrasound systems.

The patient 108 may be positioned in a supine, prone, or seated posture depending on the clinical procedure. The fingertip-mounted ultrasound imaging device 102 allows the health care professional 106 to maintain optimal imaging contact and direction, even when the procedure requires difficult access angles due to the curvature of the facial or body structures. The compact nature of the fingertip-mounted ultrasound imaging device 102 also enables bedside, emergency, or ambulatory use where conventional ultrasound probes may be impractical, allowing quick positioning, stable and accurate scanning

The display 110 may be operatively coupled to the fingertip-mounted ultrasound imaging device 102 through a wired or wireless interface. In some embodiments, the display 110 may be integrated with a computing device, such as a smartphone, tablet, laptop, or standalone console. The display 110 may present real-time ultrasound images, including tissue structures, vascular features, or foreign objects, along with optional needle or cannula trajectory overlays and measurement indicators, including Doppler flows for vascular structures identification. In some embodiments, the fingertip-mounted ultrasound imaging device 102 may communicate with the display 110 using a wireless interface, enabling cable-free operation during clinical procedures.

In certain implementations, the display 110 may be incorporated into an augmented-reality (AR) headset, enabling hands-free operation and direct visualization of both, the ultrasound image and the patient site of the procedure, in the health care professional's field of view. The display 110 may further include, but is not limited to, user interface controls for image gain, depth adjustment, Doppler imaging, measurements, annotations, or the recording of procedural data.

In operation, the health care professional 106 wears the fingertip-mounted ultrasound imaging device 102 on the index finger of the first hand. The transducer is placed against the patient's skin near the target site. The display 110 shows a live ultrasound image, allowing visualization of skin and subcutaneous tissue layers. The clinician identifies the target structure then manipulates the syringe 104 using the second hand to align the needle trajectory with the ultrasound image. As the needle or cannula advances, its movement is observed in real time on the display 110, ensuring precise positioning within the desired tissue layer or target area.

Once the needle reaches the correct depth and space, the clinician can deliver products or medications, aspirate fluid, or perform a biopsy, all while maintaining imaging feedback through the fingertip-mounted ultrasound imaging device 102. After the procedure, the images or video may be stored or transmitted for documentation or diagnostic analysis.

The example environment 100 illustrated in FIG. 1 provides multiple advantages over conventional ultrasound setups. The fingertip-mounted ultrasound imaging device 102 enables single-operator control of both imaging and intervention, reduces operator fatigue, improves spatial coordination, and increases the accuracy of needle or cannula guidance. The adjustable mounting joint of the fingertip-mounted ultrasound imaging device 102 allows the scanning plane to be dynamically altered without requiring hand repositioning, enabling optimal imaging angles across different patient anatomies and positions.

This configuration allows use in diverse applications, including ultrasonography assessment, intravascular access, regional anaesthesia, musculoskeletal injections, biopsies, abscess drainage, targeted drug delivery, and avoidance of high-risk structures, among others.

FIG. 1B illustrates another example environment 150 in which the fingertip-mounted ultrasound imaging device 102 is utilized by a health care professional 106 to perform an ultrasound-guided facial procedure on a patient 108. Similar to the embodiment shown in FIG. 1A, the environment includes a medical device 104, such as a syringe used for injecting fillers, anaesthetics, or other therapeutic substances. The display 110 is operatively coupled to the fingertip-mounted ultrasound imaging device 102, allowing for the visualization of real-time ultrasound images. In this embodiment, the healthcare professional 106 additionally wears augmented reality (AR) glasses 112 that display ultrasound images directly in the clinician's field of view.

During operation in the embodiment of FIG. 1B, the fingertip-mounted ultrasound imaging device 102 is positioned on the index finger of the health care professional's first hand, while the syringe 104 is held in the second hand. The clinician positions the ultrasound transducer of device 102 over a target region on the patient's face, such as the cheek, nasolabial fold, lips, peri-orbital area, or other aesthetic region. Real-time ultrasound imaging displayed on the display 110 or the AR glasses 112 allows the clinician to identify anatomical layers, including skin, subcutaneous fat planes, facial vasculature, and potential filler spaces.

For example, during a facial filler implant procedure, the health care professional 106 may position the ultrasound transducer of device 102 over the patient's target region of the face, identify the target area of specific depth and space using real-time ultrasound imaging, and simultaneously guide the needle or cannula toward the targeted area while continuously monitoring needle trajectory on display 110 or the AR glasses 112. This integrated approach minimises procedural time, improves accuracy, optimises treatment outcomes, and enhances safety compared to traditional ultrasound systems.

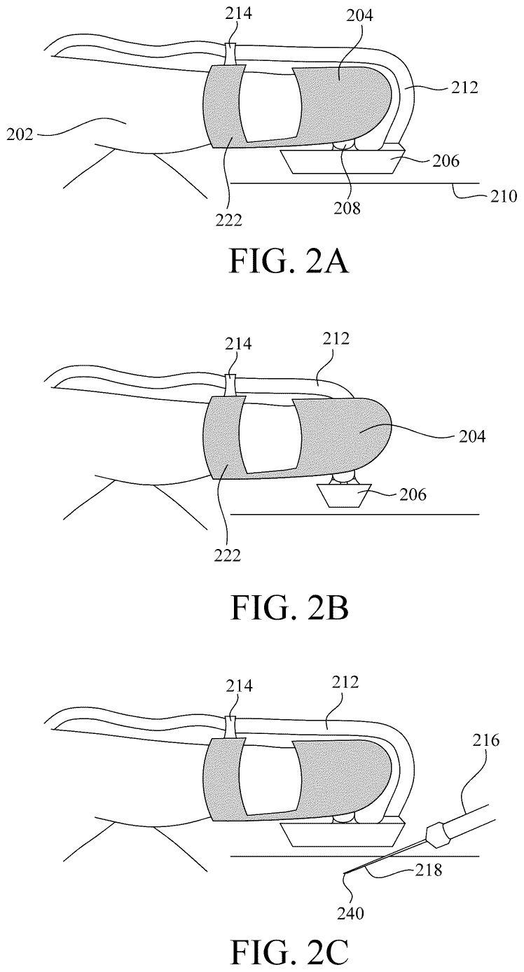

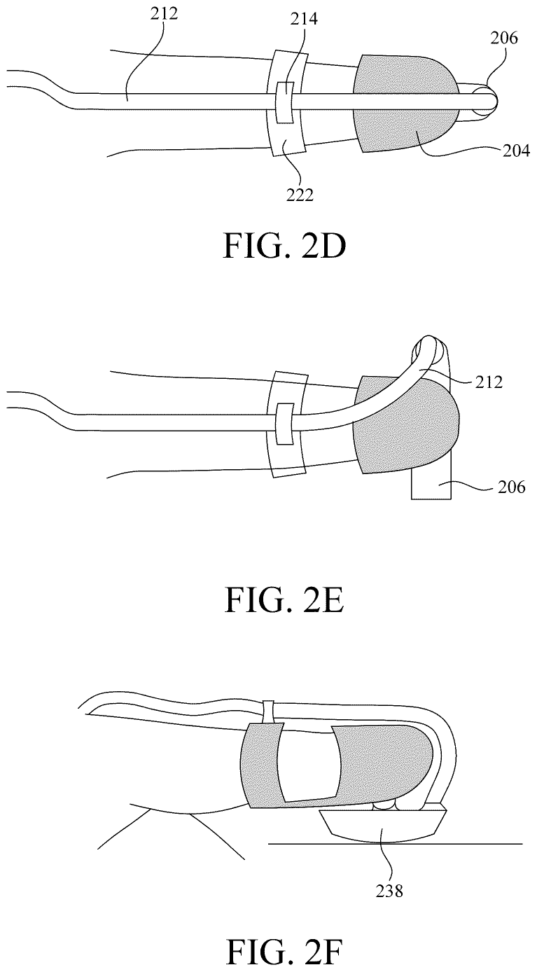

FIGS. 2A-2C illustrate example configurations of a fingertip-mounted ultrasound imaging device 102 worn on a finger 202 in accordance with some embodiments of the present disclosure.

As shown, the fingertip-mounted ultrasound imaging device 102 comprises a housing 204 secured to the ventral surface of the user's finger 202 by a fastening mechanism 222, such as a strap, elastic band, or hook-and-loop fastener. The housing 204 may include a contoured inner surface and a soft liner that conforms to the finger anatomy and provides comfort, grip, and tactile feedback.

An ultrasound transducer 206 is coupled to the housing 204 via an adjustable mounting joint 208. In some embodiments, the adjustable mounting joint 208 allows the ultrasound transducer 206 to move pivotably, tiltable, or rotatably about one or more spatial axes, thereby altering the orientation of the scanning plane relative to the user's finger 202. The adjustable mounting joint 208 may include a pivot point defining the axis of rotation, a tilt range, and a rotation range.

In some embodiments, the adjustable mounting joint 208 may be implemented as a ball-and-socket joint, universal joint, or hinge-type joint, and may incorporate a frictional or detent locking mechanism to maintain a selected angle. The adjustable mounting joint 208 may further include a spring-biased resistance that provides smooth movement and tactile feedback when the user repositions the ultrasound transducer 206.

In some embodiments, the ultrasound transducer 206 may include an array of transducer elements enclosed in a biocompatible and waterproof casing. The array may be linear, curved, or convex, and is configured to emit ultrasound waves into tissue and receive echo signals for real-time image generation. In some embodiments, the ultrasound transducer 206 may operate in any range within the frequency range of approximately 7 MHz to 50 MHz, depending on the indication the finger probe is designed for.

In some embodiments, the ultrasound transducer 206 may also support advanced imaging functions such as Doppler flow imaging, beam steering, and compound imaging or other advance features. In some embodiments, a sterile coupling pad or ultrasound gel may be applied between the ultrasound transducer 206 and the patient's skin 210 to improve acoustic coupling. In clinical environments, a disposable sterile sheath may cover the entire fingertip device 102 for aseptic use.

In some embodiments, a power and data cable 212 connects the ultrasound transducer 206 to the display 110 or computing unit. The power and data cable 212 may be routed through a cable guide 214 integrated into the fastening strap or the housing 204, running along the dorsal surface of the finger 202 to prevent interference with the user's field of operation. In some other embodiments, the computing unit may communicate wirelessly with a display module worn on the wrist or forearm, or with a pair of AR Glasses, transmitting signals via Wi-Fi, Bluetooth, or a proprietary low-latency protocol. In certain embodiments, the fingertip-mounted ultrasound imaging device 102 may include an onboard power source and a wireless communication module that transmits ultrasound data to a remote computing or display unit, thereby eliminating the need for a physical power and data cable.

As depicted in FIG. 2A, the ultrasound transducer 206 is oriented substantially parallel to the finger 202. This configuration produces a scanning plane 210 or the patient's skin 210 that is also parallel to the finger axis. The health care professional 106 may gently press the fingertip-mounted transducer against the patient's skin 210 to maintain coupling, while the adjustable mounting joint 208 allows subtle angular adjustments in the parallel axis.

In FIG. 2B, the ultrasound transducer 206 has been rotated via the adjustable mounting joint 208 into a perpendicular orientation relative to the finger 202. In this position, the scanning plane 210 is orthogonal to the finger axis, providing a cross-sectional or transverse view of the underlying tissue. This configuration is advantageous, providing a more natural and ergonomic angle for the second hand to handle and deliver the medical devices, such as needles or cannulas.

In FIG. 2C, the fingertip-mounted ultrasound imaging device 102 is shown in use during a clinical procedure. The ultrasound transducer 206 is positioned over a patient's skin 210 at a target site 240, imaging a tissue layer beneath the skin. The medical device 104, such as a syringe 216 with a needle or cannula 218, is introduced toward the target site under real-time ultrasound guidance. The health care professional 106 manipulates the syringe 216 with one hand while the other hand holds the fingertip-mounted imaging device 102, as shown in subsequent figures.

The adjustable mounting joint 208 allows the scanning plane 210 to be dynamically altered in order to obtain the most optimal plane, enabling the clinician to track the needle 218 in either an in-plane orientation, where the needle trajectory aligns with the scanning plane, or an out-of-plane orientation, where the needle crosses the scanning plane at an angle. This capability permits visualization of the needle tip regardless of approach angle, improving precision and safety during injections, aspirations, or catheter placements.

In some embodiments, the housing 204 may be constructed from medical-grade polymeric materials such as polyurethane, silicone, or thermoplastic elastomers, capable of withstanding sterilization by autoclave, ethylene oxide, or disinfectant wipes.

FIGS. 2D-2F illustrate additional embodiments and operational configurations of the fingertip-mounted ultrasound imaging device 102 in accordance with some embodiments of the present disclosure. As shown in FIGS. 2D-2F, the FIGS. 2D-2F focuses on cable routing, transducer orientation control, and ergonomic adaptability for various finger postures and patient access angles.

As shown in FIG. 2D, the power and data cable 212 is operatively coupled to the ultrasound transducer 206 and routed along the dorsal side of the user's finger 202 through an integrated cable guide 214. The cable guide 214 may be formed as a flexible loop, O-ring, or clip mounted to the fastening mechanism 222, such as the fastening strap of the housing 204. This configuration secures the power and data cable 212 close to the finger surface and prevents it from interfering with the clinician's working area or obstructing the manipulation of medical instruments. The cable guide 214 allows controlled movement of the data cable 212.

In some embodiments, the power and data cable 212 may be configured to transmit both electrical drive signals and echo signal data between the ultrasound transducer 206 and a connected computing or display unit 110. Further, in some embodiments, the power and data cable 212 may include shielding to minimize electromagnetic interference and ensure signal integrity during imaging. In some embodiments, the power and data cable 212 may include an integrated strain relief section at the transducer interface to reduce mechanical stress during flexion of the finger or rotation of the ultrasound transducer 206.

FIG. 2E depicts an embodiment in which the transducer module 208 is angled perpendicular to the finger axis via the adjustable mounting joint 208, demonstrating how the joint and cable guide cooperate to maintain a stable connection as the scanning plane changes.

In this orientation, the scanning plane is angled relative to the finger axis to align with the user's optimal axis and, if present, the needle or cannula's axis. The power and data cable 212 flexes naturally along the curved path of the cable guide without tension, enabling smooth, continuous adjustment of the transducer orientation.

In some embodiments, the adjustable mounting joint 208 may include an internal pivot bearing or a semi-rigid elastomeric joint segment that provides multi-axis flexibility. The adjustable mounting joint 208 may allow angular displacement of approximately 45 degrees in tilt and 270 degrees in rotation, as limited by the data cable 212. This flexible coupling allows the clinician to maintain optimal contact between the ultrasound transducer 206 and the patient's skin 210 even as the finger or hand posture changes.

The embodiment as shown in FIG. 2E is useful for imaging scenarios in which the operator must reach over or around an anatomical feature at an angle other than perpendicular. The dynamic range of movement provided by the adjustable mounting joint 208 and the secure cable routing ensures that the ultrasound transducer 206's face remains optimally acoustically coupled and stable.

FIG. 2F depicts another embodiment of the fingertip-mounted ultrasound imaging device 102, incorporating a curved or convex transducer probe 238 in accordance with some embodiments of the present disclosure.

As shown in FIG. 2F, the transducer probe 238 is mounted to the adjustable mounting joint 208 and is configured to generate a curved scanning field suitable for imaging anatomical regions requiring a wider or deeper field of view. The convex or curved configuration enhances visualization of underlying tissue layers that are not readily captured using a flat or linear array.

The curved or convex transducer probe 238 operates in substantially the same manner as the linear probe embodiments shown in FIGS. 2A-2E, emitting ultrasound waves into the tissue and receiving reflected echo signals for real-time image generation. The adjustable mounting joint 208 permits the curved transducer probe 238 to be pivoted, tilted, or rotated to achieve optimal contact and image orientation relative to the patient's skin surface.

In some implementations, the curved transducer probe 238 may be preferred for applications involving deeper tissue targets or broader anatomical mapping, such as abdominal imaging, musculoskeletal assessment, or regional anaesthesia, where a wider imaging footprint is beneficial. The curved geometry enables the clinician to visualize superficial and deeper structures within the same image frame, enhancing procedural efficiency.

The curved or convex transducer probe 238, as shown in FIG. 2F, may therefore be used interchangeably with a linear transducer probe in the embodiments demonstrated in FIGS. 2A-2E, without altering the functional or ergonomic characteristics of the fingertip-mounted ultrasound imaging device. This modularity allows the system to accommodate multiple probe types, optimizing performance for various procedural requirements and anatomical locations.

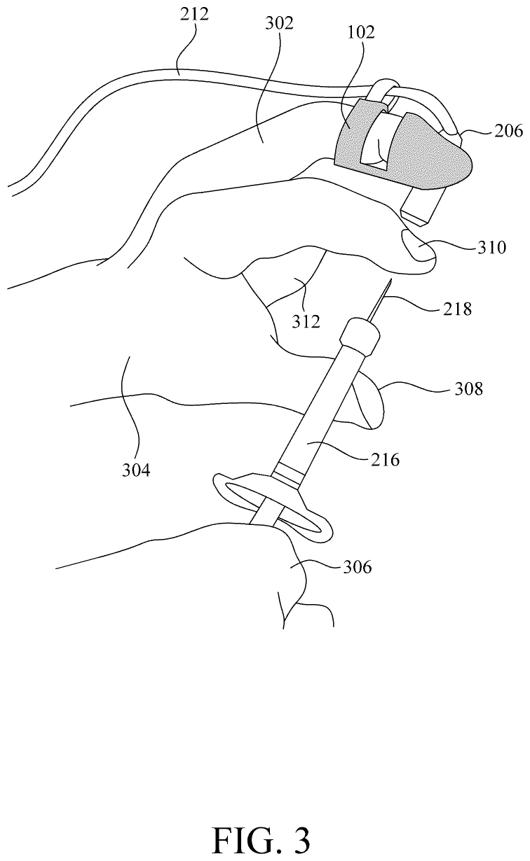

FIG. 3 illustrates an example operational configuration demonstrating how a clinician employs the fingertip-mounted ultrasound imaging device 102 in coordination with the medical device 104, in accordance with some embodiments of the present disclosure. FIG. 3 depicts the hand posture and relative positioning of the two devices during an ultrasound-guided injection or aspiration procedure.

The fingertip-mounted ultrasound imaging device 102 is secured to a middle finger 302 of the clinician's first hand 304. The fingertip-mounted ultrasound imaging device 102 includes the housing 204, the adjustable mounting joint 208, and the ultrasound transducer 206. The adjustable mounting joint 208 is configured to permit pivoting, tilting, or rotational movement of the ultrasound transducer 206 relative to the housing 204, enabling dynamic reorientation of the scanning plane during a procedure. Further, as shown in FIG. 3, the power and data cable 212 extends from the ultrasound transducer 206, connecting to an external processing and display unit (not shown in FIG. 3).

The second hand 306 of the health care professional 106 holds and manipulates the syringe 216, while the first hand 304 provides additional support and stabilization. The thumb 308, an index finger 310, and a palm 312 of the first hand 304. The thumb 308 is used to support the syringe, while the middle finger 302, wearing the fingertip-mounted ultrasound device 102, positions the ultrasound transducer 206 against the patient's skin surface at a target site. This coordinated configuration allows the clinician to maintain simultaneous control of imaging and injection with both hands.

The ultrasound transducer 206 emits ultrasound waves into the tissue beneath the target site and receives reflected echo signals that are processed to generate a real-time ultrasound image. This image is displayed on a connected display unit 110 (as illustrated previously in FIG. 1), providing continuous visualization of both the anatomical target and the advancing needle or cannula 218.

In operation, the clinician places the ultrasound transducer 206 in contact with the patient's skin and gently adjusts its orientation using the adjustable mounting joint 208 to achieve optimal visualization of the target structure. The adjustable mounting joint 208 allows the scanning plane to be dynamically altered without requiring repositioning of the clinician's wrist or forearm, thus maintaining ergonomic comfort and image stability.

The clinician then advances the syringe 216 using the second hand 306, guiding the needle 218 toward the target site. Throughout the advancement, the clinician observes the needle 218 on the ultrasound display 110. Depending on the approach, the clinician may employ either an in-plane technique, where the needle path lies along the ultrasound scanning plane, allowing visualization of the entire needle shaft, or an out-of-plane technique, where the needle intersects the scanning plane perpendicularly, appearing as a bright dot corresponding to the needle tip.

The fingertip-mounted ultrasound device 102 enables continuous image acquisition even when the syringe or the clinician's hand changes position. This stability allows for micro-adjustments of the ultrasound transducer 206 during the injection, helping the clinician maintain visualization of the target structure and needle tip at all times. The real-time feedback facilitates accurate placement of the needle or cannula into the correct target tissue beneath or within the skin.

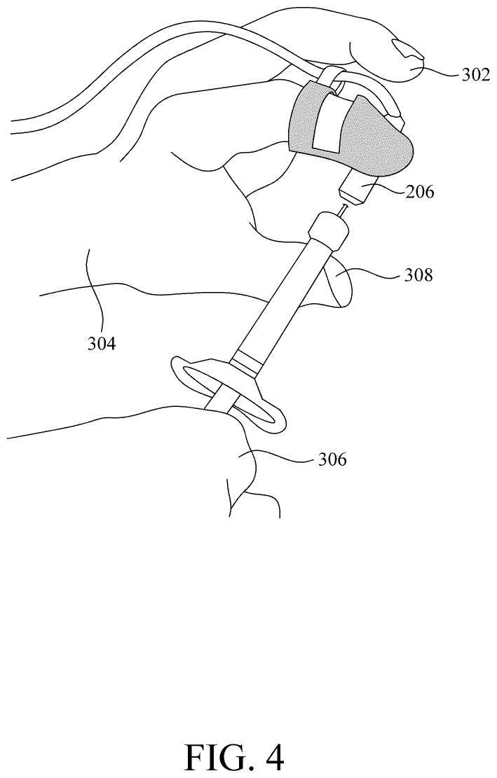

FIG. 4 illustrates another embodiment of the system, depicting the use of a fingertip-mounted ultrasound imaging device 102 during an ultrasound-guided injection procedure in accordance with some embodiments of the present disclosure.

As shown in FIG. 4, the fingertip-mounted ultrasound imaging device 102 is secured to an index finger 310 of a first hand 304 of a clinician or health care professional. The fingertip-mounted ultrasound imaging device 102 includes the housing 204, the adjustable mounting joint 208, and the ultrasound transducer 206 coupled to the joint. Housing 204 is contoured to fit the ventral surface of index finger 310 and includes a fastening strap or band to ensure secure, ergonomic placement. The adjustable mounting joint 208 allows the ultrasound transducer 206 to pivot, tilt, or rotate in multiple axes, thereby adjusting the orientation of the scanning plane relative to the patient's skin.

The medical device 104, shown in FIG. 4 as the syringe 216 having the needle or cannula 218, is held in the second hand 306 of the clinician. The first hand 304, wearing the fingertip-mounted ultrasound imaging device 102 on the index finger 310, allows the middle finger 302 and the thumb 308 to stabilize the patient's skin and also assist in the stabilization of the index finger 310 and the transducer 206. The index finger 310, fitted with the fingertip-mounted ultrasound imaging device 102, positions the ultrasound transducer 206 directly over the target site on the patient's skin. This arrangement enables the clinician to maintain continuous imaging visualization while advancing the syringe needle 218 with precision.

The ultrasound transducer 206 emits acoustic waves into the tissue beneath the target site and receives reflected echo signals. These signals are processed in real time to generate an ultrasound image displayed on an external display unit 110 (not shown in this figure, see FIG. 1). The clinician observes the ultrasound image to visualize both the underlying anatomical structure and the advancing needle or cannula 218. This configuration enables real-time alignment between the needle or cannula trajectory and the ultrasound scanning plane, allowing the clinician to confirm the correct needle or cannula position before carrying out the intended procedure such as administering medication, aspirating fluid or biopsy.

The embodiment of FIG. 4 demonstrates an alternate ergonomic orientation in which the ultrasound transducer 206 is angled in line relatively to the syringe 216. The adjustable mounting joint 208 allows the clinician to dynamically maintain the scanning plane without moving the wrist or arm, maintaining stable imaging contact even during active needle advancement.

The technique illustrated in FIG. 4 permits the clinician to maintain continuous ultrasound visualization while advancing and manipulating the syringe 216. The fingertip-mounted configuration enhances tactile precision and allows simultaneous control of imaging and intervention by a single operator. This eliminates the need for an assistant to hold a separate probe, reduces procedural time, and minimizes operator fatigue. This also allows the use of several fingers, the thumb and the palm from the first hand 304 to stabilize the patient, the transducer and handle the skin surface, at the same time scanning with the ultrasound transducer 206. The clinician's natural hand posture enables seamless coordination between the imaging device and the medical instrument, particularly in confined anatomical regions or when the patient's position restricts maneuverability.

In clinical use, this alternate configuration supports both in-plane and out-of-plane ultrasound-guided techniques. In an in-plane approach, the needle or cannula 218 is advanced parallel to the scanning plane, allowing visualization of the full needle shaft and tip. In an out-of-plane approach, the needle crosses the scanning plane at an angle, appearing as a bright reflection or dot corresponding to the needle tip. The fingertip-mounted design allows fine angular adjustments of the ultrasound transducer 206 via the joint 208 to track the needle tip and or shaft continuously throughout its advancement.

The embodiment of FIG. 4 thus represents a variation of the configuration shown in FIG. 3, highlighting an alternative posture and procedural workflow that maintains the ergonomic advantages of the fingertip-mounted ultrasound imaging system. The described configuration enhances operator dexterity, supports real-time imaging alignment, and provides adaptability across a range of clinical procedures.

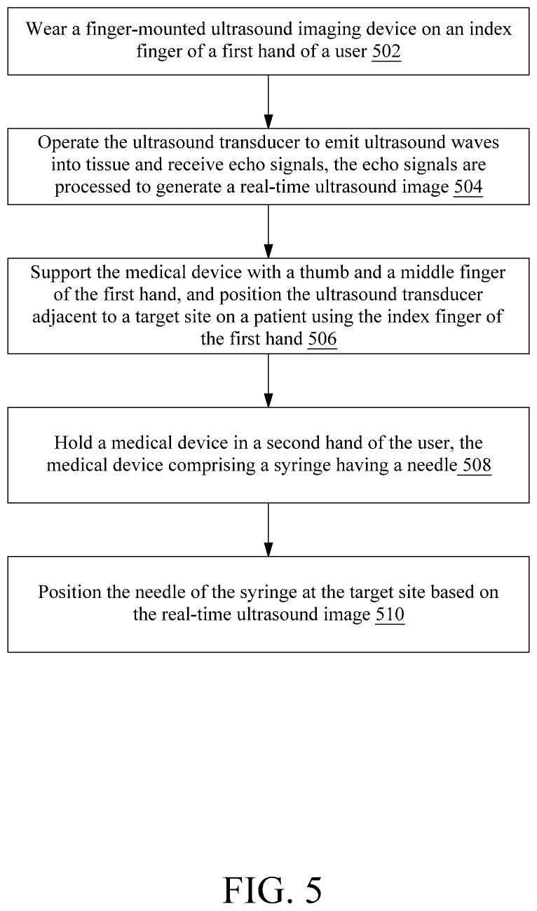

FIG. 5 illustrates an example method 500 for performing an ultrasound-guided medical procedure utilizing a fingertip-mounted ultrasound imaging device in accordance with some embodiment of the present disclosure. The method enables a clinician to maintain real-time ultrasound visualization while performing a procedure using natural and coordinated hand movements.

The method 500 includes a sequence of steps 502-510, described in greater detail below. Each step corresponds to a physical action or functional operation performed by the clinician with the fingertip-mounted ultrasound imaging device, as depicted in the earlier figures.

At step 502, the clinician wears a fingertip-mounted ultrasound imaging device on an index finger of a first hand. The fingertip-mounted ultrasound imaging device includes a housing, an adjustable mounting joint, and an ultrasound transducer coupled to the joint. The housing is secured on the ventral side of the finger using a fastening strap or elastic band.

The adjustable mounting joint permits pivotable, tiltable, and rotatable movement of the ultrasound transducer relative to the finger, enabling dynamic adjustment of the scanning plane. This configuration allows the clinician to orient the transducer accurately over the target site, even with a difficult plane of the skin due to specific face/body surface features. The fingertip-mounted form factor enhances tactile control, enabling intuitive adjustment of imaging parameters via fine finger movements rather than gross probe repositioning. The user acquires the desired image of the target area by making further fine adjustments to the transducer.

In some embodiments, the device may include a cable guide that routes a power and data cable along the dorsal surface of the finger, maintaining a clutter-free workspace. Alternatively, the device may operate wirelessly, powered by a wearable data processing and display module attached to the wrist or forearm.

At step 504, the clinician operates the ultrasound transducer to emit ultrasound waves into tissue and receive corresponding echo signals. The echo signals are processed to generate a real-time ultrasound image, which is displayed on an external monitor, a mobile device, or an augmented reality (AR) headset.

The ultrasound transducer may operate in a frequency range of 7 MHz to 50 MHz. In some embodiments, the transducer supports B-mode, Doppler, and compound imaging modes. The clinician may adjust parameters such as gain, depth, or focus using either on-device touch controls or a linked computing interface.

During this step, the clinician dynamically adjusts the transducer's orientation using the adjustable mounting joint. By pivoting or tilting the joint, the scanning plane can be realigned to track the advancing needle or cannula during the procedure. The joint's frictional resistance or detent mechanism ensures that the selected orientation remains stable once set.

At step 506, the clinician holds a medical device in their second hand. In the embodiment shown, the medical device comprises a syringe with a needle or cannula, though other instruments, such as a catheter, biopsy needle, or aspiration device, may also be used.

The second hand is responsible for primary manipulation of the syringe, including plunger operation and depth control. However, as shown in FIG. 3, the first hand (the hand wearing the ultrasound device) also provides stabilization and support to the syringe. This two-hand coordination enables simultaneous imaging and needle or cannula manipulation by a single operator, eliminating the need for an assistant and ensuring precise procedural control.

At step 508, the clinician holds the medical device with the second hand and supports it with the thumb of the first hand. This step establishes the key ergonomic advantage of the system. The clinician's first hand thus acts as a dual-purpose control point: one part stabilizes the medical instrument, and the other part maintains the imaging field of view.

The target site may correspond to any tissue structure, depending on the type of procedure. The transducer's adjustable joint allows the clinician to fine-tune the scanning plane so that both the target site and the anticipated needle path are clearly visualized on the connected display.

At step 510, the clinician positions the needle or cannula of the syringe at the target site based on the real-time ultrasound image obtained in step 408. As the clinician advances the needle toward the target, the ultrasound image provides continuous visualization of both the anatomical structure and the needle or cannula tip.

Depending on the procedural technique, the clinician may employ an in-plane approach, in which the needle or cannula trajectory aligns with the ultrasound scanning plane, allowing full-length visualization of the needle or cannula shaft and tip; or an out-of-plane approach, in which the needle or cannula crosses the scanning plane perpendicularly, appearing as a bright point or dot on the display.

Once the needle or cannula tip reaches the desired tissue target, the clinician can perform the intended procedure, such as injecting medication, aspirating fluid, performing a biopsy, or inserting a catheter, while maintaining imaging confirmation throughout. The fingertip-mounted device allows the clinician to make minor transducer adjustments without disrupting needle or cannula alignment or hand positioning.

The above description of the shown example implementations, including what is described in the Abstract, is not intended to be exhaustive or to limit the implementations to the precise forms disclosed. Although specific implementations of and examples are described herein for illustrative purposes, various equivalent modifications can be made without departing from the spirit and scope of the disclosure, as will be recognized by those skilled in the relevant art. Moreover, the various example implementations described herein may be combined to provide further implementations.

1. A finger-mounted ultrasound imaging device comprising:
a housing configured to be worn by a user, the housing including a contoured inner surface and a fastening mechanism adapted to secure the housing to a user;
an adjustable mounting joint operatively coupled to the housing, the adjustable mounting joint configured to permit at least one of pivotable, tiltable, and rotatable movement in one or more spatial axes; and
an ultrasound transducer operatively coupled to the adjustable mounting joint, the ultrasound transducer configured to emit ultrasound waves into tissue and receive corresponding echo signals for real-time image generation,
wherein the adjustable mounting joint is configured to permit pivoting, tilting, and rotation of the ultrasound transducer relative to the housing to selectively alter an angle of the ultrasound transducer relative to a longitudinal axis of a middle finger while maintaining the housing attached to the middle finger.

2. The device of claim 1, wherein the adjustable mounting joint comprises at least one of a ball-and-socket joint, a universal joint, and a hinge joint.

3. The device of claim 1, wherein the ultrasound transducer comprises at least one of a linear probe, a curved probe, and a convex probe.

4. The device of claim 1, wherein the housing is configured to be worn on the middle finger of a first hand of the user, wherein the ultrasound transducer is configured to be stabilized by the first hand while manipulating the patient's skin for obtaining optimized imaging.

5. The device of claim 1, wherein the housing further comprises a fastening strap configured to secure the device proximal to the distal finger joint while permitting limited flexion of the distal finger joint.

6. The device of claim 5, further comprising a cable management guide operatively coupled to one of the housing and the fastening strap, the cable management guide configured to route a data cable, which contains numerous smaller cables, along a dorsal surface of the finger.

7. The device of claim 1, wherein ultrasound images generated by the ultrasound transducer are displayed in real time on at least one of a smartphone, a tablet, a computer, AR Glasses, and a standalone ultrasound console.

8. An ultrasound imaging system comprising:
a finger-mounted ultrasound imaging device;
a power and data unit configured to supply electrical drive signals to an ultrasound transducer and to receive echo signal data generated by the ultrasound transducer;
a computing device communicatively coupled to the power and data unit, the computing device configured to process the echo signals into ultrasound images; and
a display device operatively coupled to the computing device and configured to present the ultrasound images in real time, wherein the finger-mounted ultrasound imaging device comprises:
a housing configured to be worn by a user; and
an adjustable mounting joint operatively coupled to the housing and configured to permit pivoting, tilting, and rotation of the ultrasound transducer relative to the housing to selectively alter an angle of the ultrasound transducer relative to a longitudinal axis of a middle finger while maintaining the housing attached to the middle finger.

9. The ultrasound imaging system of claim 8, wherein:
the housing including a contoured inner surface and a fastening mechanism adapted to secure the housing to a user;
the adjustable mounting joint configured to permit at least one of pivotable, tiltable, and rotatable movement in one or more spatial axes; and
an ultrasound transducer operatively coupled to the adjustable mounting joint, the ultrasound transducer configured to emit ultrasound waves into tissue and receive corresponding echo signals for real-time image generation,
wherein the adjustable mounting joint is configured to provide a variable orientation of the ultrasound transducer to dynamically alter or optimize a scanning plane.

10. The ultrasound imaging system of claim 8, wherein the finger-mounted ultrasound imaging device, the power and data unit, the computing device, and the display device are manufactured to be combined into a single unit or multiple units with a varying combination of subunits of the ultrasound imaging system.

11. A method of performing an ultrasound-guided medical procedure, the method comprising:
wearing a finger-mounted ultrasound imaging device on an index finger of a first hand of a user, the fingertip-mounted ultrasound imaging device comprising a housing, an adjustable mounting joint coupled to the housing, and an ultrasound transducer operatively coupled to the adjustable mounting joint;
holding a medical device in a second hand of the user, the medical device comprising a syringe having a needle or cannula;
positioning the ultrasound transducer over the target site on a patient using the index finger of the first hand;
operating the ultrasound transducer to emit ultrasound waves into tissue and receive echo signals, the echo signals are processed to generate a real-time ultrasound image; and
supporting the medical device with a thumb of the first hand, and positioning the needle of the syringe aiming at the target site based on the real-time ultrasound image, wherein the adjustable mounting joint is configured to-permit pivoting, tilting, and rotation of the ultrasound transducer relative to the housing to selectively alter an angle of the ultrasound transducer relative to a longitudinal axis of the index finger while maintaining the housing attached to the index finger.

12. The method of claim 11, wherein the real-time ultrasound image displays the position and direction of the needle trajectory to assist the user in guiding the needle of the syringe toward the target site.

13. The method of claim 11, wherein the target site comprises a tissue layer of the skin and beneath the skin of the patient.

14. The method of claim 11, further comprising adjusting an orientation of the ultrasound transducer using the adjustable mounting joint to dynamically alter a scanning plane and axis.

15. The method of claim 11, wherein the adjustable mounting joint comprises at least one of a ball-and-socket joint, a universal joint, and a hinge joint.

16. The method of claim 11, wherein the ultrasound transducer comprises at least one of a linear transducer, a curved transducer, and a convex transducer.

17. The method of claim 11, wherein an operator manipulates the skin while stabilizing the ultrasound transducer and making adjustments in position and plane, in order to achieve the optimal im
